(19) United States
(12) Patent Application Publication
(10) Pub. No.: US 2023/0123140 A1
(43) Pub. Date: Apr. 20, 2023

(54) DOSE TREATMENTS OF TAUOPATHIES

(71) Applicant: AC Immune SA, Lausanne (CH)

(72) Inventors: Sonia Maria Poli, Onex (CH); Francesca Capotosti, Penthalaz (CH)

(21) Appl. No.: 17/912,138

(22) PCT Filed: Mar. 18, 2021
(86) PCT No.: PCT/EP2021/057025

§ 371 Date: Sep. 16, 2022

Related U.S. Application Data
(60) Provisional application No. 62/991,770, filed on Mar. 19, 2020.

(51) Int. Cl. A61K 31/5377

(52) U.S. Cl. CPC A61K 31/5377

(57) ABSTRACT

The present invention relates to the use of ACI-3024 in the treatment, alleviation or prevention in humans of a group of disorders and abnormalities associated with Tau protein aggregates, such as Alzheimer's disease (AD) and progressive supranuclear palsy (PSP).

### FIELD OF THE INVENTION

The present invention relates to the use of ACI-3024 in the treatment, alleviation or prevention of a group of disorders and abnormalities associated with Tau protein aggregates, such as Alzheimer's disease (AD), progressive supranuclear palsy (PSP), frontotemporal lobar degeneration with predominant Tau pathology (FTLD-Tau) including frontotemporal lobar degeneration caused by a MAPT gene mutation (FTLD-MAPT).

### BACKGROUND OF THE INVENTION

Alzheimer's disease (AD) is a neurological disorder primarily thought to be caused by amyloid plaques, an extracellular accumulation of abnormal deposit of (amyloid-beta, Abeta) Aβ aggregates in the brain. The other major neuropathological hallmarks in AD are the intracellular neurofibrillary tangles (NFT) that originate by the aggregation of the hyperphosphorylated Tau protein, misfolded Tau or pathological Tau and its conformers. AD shares its etiopathology with many neurodegenerative tauopathies, in particular with specified types of frontotemporal dementia (FTD). The Tau protein is a freely soluble, “naturally unfolded” protein that binds avidly to microtubuli (MT) to promote their assembly and stability. MT are of major importance for the cytoskeletal integrity of neurons—and thereby for the proper formation and functioning of neuronal circuits, hence for learning and memory. The binding of Tau to MT is controlled by dynamic phosphorylation and de-phosphorylation, as demonstrated mainly in vitro and in non-neuronal cells. In AD brain, Tau pathology develops later than amyloid pathology, but it is still discussed controversially if Aβ protein is the causative agent in AD which constitutes the essence of the so-called amyloid cascade hypothesis (Hardy et al., Science 1992, 256, 184-185; Musiek et al., Nature Neurosciences 2015, 18(6), 800-806). The exact mechanisms that link amyloid to Tau pathology remain largely unknown, but are proposed to involve activation of neuronal signaling pathways that act on or by GSK3 (Glykogensynthase-Kinase 3) and cdk5 (Cell division protein kinase 5) as the major “Tau-kinases” (Muyllaert et al., Rev. Neurol. (Paris), 2006, 162, 903-7; Muyllaert et al., Genes Brain and Behav. 2008, Suppl 1, 57-66). Even if the tauopathy develops later than amyloid, it is not just an innocent side-effect but a major pathological executer in AD. In experimental mouse models the cognitive defects caused by amyloid pathology are nearly completely alleviated by the absence of Tau protein (Roberson et al., Science, 2007, 316(5825), 750-4) and the severity of cognitive dysfunction and dementia correlates with the tauopathy, not with amyloid pathology.

Diseases involving Tau aggregates are generally listed as tauopathies and they include, but are not limited to, Alzheimer's disease (AD), familial AD, PART (primary age-related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy including Lafora disease, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease. (Williams et al., Intern. Med. J., 2006, 36, 652-60; Kovacs et al., J Neuropathol Exp Neurol. 2008; 67(10): 963-975; Higuchi et al., Neuropsychopharmacology—5th Generation of Progress, 2002, Section 9, Chapter 94: 1339-1354; Hilton et al., Acta Neuropathol. 1995; 90(1):101-6; Iqbal et al., Biochimica et Biophysica Acta 1739 (2005) 198-210; McQuaid et al., Neuropathol Appl Neurobiol. 1994 April; 20(2):103-10; Vossel et al., Lancet Neurol 2017; 16: 311-22; Stephan et al., Molecular Psychiatry (2012) 17, 1056-1076; Anderson et al., Brain (2008), 131, 1736-1748; Savica et al., JAMA Neurol. 2013; 70(7):859-866; Brown et al. Molecular Neurodegeneration 2014, 9:40; El Khoury et al., Front. Cell. Neurosci., 2014, Volume 8, Article 22: 1-18; Tanskanen et al., Ann. Med. 2008; 40(3):232-9; Gupta et al., CAN J OPHTHALMOL-VOL. 43, NO. 1, 2008: 53-60; Dickson et al., Int J Clin Exp Pathol 2010; 3(1):1-23; Fernandez-Nogales et al., Nature Medicine, 20, 881-885 (2014); Bi et al., Nature Communications volume 8, Article number: 473 (2017); Murray et al., Biol Psychiatry. 2014 Apr. 1; 75(7): 542-552).

Of all the agents in clinical trials for the treatment of Alzheimer's disease in 2019, the ones targeting Tau are very scarce and represent only 18% of the clinical trials, this correspond to 17 molecules of which only 7 are small molecules. LMTX (Methylene Blue, methylthioninium, also known as TRx0237 and LMTM) is listed as an anti-Tau agent in phase 3 (Cummings et al., Alzheimer's disease drug development pipeline: 2019; Alzheimer's & Dementia: Translational Research & Clinical Interventions 5 (2019) 272-293). LMTX is a purified form of Methylene Blue and Methylene Blue has been reported to interact with Abeta aggregates leading to Aβ clearance in transgenic AD mouse models (Medina et al. Methylene blue reduces Aβ levels and rescues early cognitive deficit by increasing proteasome activity. Brain Pathol. 21 (2011) 140-149), Other studies emphasize a generalized anti-aggregation effect for Methylene Blue against aggregation-prone proteins, such as prion protein (Cavaliere et al. Binding of methylene blue to a surface cleft inhibits the oligomerization and fibrillization of prion protein. Biochim Biophys Acta. 32 (2013) 20-28) and TDP-43 (Arai et al. Phosphorylated and cleaved TDP-43 in ALS, FTLD and other neurodegenerative disorders and in cellular models of TDP-43 proteinopathy. Neuropathology. 30 (2010) 170-181). In mice overexpressing human α-synuclein, LMTM treatment reduced α-synuclein inclusions in the brain, and normalized movement and anxiety-related behaviors (Schwab et al. A protein aggregation inhibitor, Leuco-Methylthioninium Bis(Hydromethanesulfonate), decreases α-synuclein inclusions in a transgenic mouse model of synucleinopathy. Front Mol Neurosci. 10 (2018) 447, doi: 10.3389/fnmol.2017.00447).

Current therapeutic approaches that target Tau protein comprise mainly antibody-based approaches with the main limitation of targeting only extracellular Tau. Among the approaches using small molecules, several Tau kinase inhibitors have been developed, despite being very challenging with respect to toxicity and specificity. Nevertheless, currently only one kinase inhibitor, Nilotinib, is tested in clinical trials.

WO2019/134978A1 refers to specific compounds which are suitable for treating Tauopathies. The compound ACI-3024 described in WO2019/134978A1 (a) displays high capability in decreasing Tau aggregates by recognizing aggregated Tau and disaggregating Tau, for example by changing the Tau aggregate molecular conformation, and/or (b) prevents the formation of Tau aggregates, and/or (c) interferes intracellularly with Tau aggregates, and/or (d) reduces Tau misfolding and hyperphosphorylation in vivo and/or (e) reduces neuroinflammatory markers. While not wishing to be bound by theory, it is assumed that the compound ACI-3024 inhibits the Tau aggregation or disaggregate preformed Tau aggregates including when present intracellularly. Due to their unique design features, this compound displays properties such as appropriate lipophilicity and molecular weight, brain uptake and pharmacokinetics, cell permeability, solubility and metabolic stability, in order to be a successful medicament for the treatment, alleviation or prevention of tauopathies.

It was surprisingly found that the plasma half-life of ACI-3024 in humans was very long (>24 h), and the CSF concentration of ACI-3024 were surprisingly high.

### SUMMARY OF THE INVENTION

The present invention is summarized in the following items:

A compound ACI-3024 for use in the treatment, prevention or alleviation of a disorder or an abnormality associated with Tau protein aggregates or a tauopathy comprising administering said compound at an amount up to 1500 (one thousand five hundred) mg/day to a mammal subject in need thereof.

A compound ACI-3024 for use in the treatment, prevention or alleviation of a disorder or an abnormality associated with Tau protein aggregates or a tauopathy comprising administering said compound to a subject in need thereof at an amount that results in 30 nM minimal predose level (Cmin) of the compound in CSF of the subject.

A compound ACI-3024 for use in the treatment, prevention or alleviation of a disorder or an abnormality associated with Tau protein aggregates or a tauopathy comprising administering to a mammal subject said compound at an amount that results in a concentration above or equal to 30 nM of said compound in CSF of the subject, prior to administering any new dose of said compound to the subject (ie minimal predose level). Specifically, the ACI-3024 amount that is administered to a subject in a first dose results in a minimum of 30 nM concentration in the patient's CSF (and such concentration is measured) prior to administering a second dose. The compound is administered to a mammal subject said compound at an amount that results in a Cmin concentration above or equal to 30 nM of said compound in CSF of the subject. Also, embodiments of the present disclosure include predicting or measuring the patient's CSF to ensure that the concentration is above or equal to 30 nM of said compound, and when the concentration is near to dropping below that threshold—administering one or more additional dose(s) of the compound.

A method for treating, preventing or alleviating a disorder or an abnormality associated with Tau protein aggregates by administering ACI-3024 at an amount up to 1500 mg/day to a mammal subject in need thereof.

The compound ACI-3024 is preferably administered in a range of 25 mg/day to 1500 mg/day and the preferred amounts are selected from 25, 100, 300, 350, 400, 450, 500, 550, 750, 1000, 1400 and 1500 mg/day.

The preferred disorder or the abnormality associated with Tau protein aggregates as mentioned above are selected from Alzheimer's disease (AD) progressive supranuclear palsy (PSP), frontotemporal lobar degeneration with predominant Tau pathology (FTLD-Tau) including frontotemporal lobar degeneration caused by a MAPT gene mutation (FTLD-MAPT).

A method for decreasing Tau aggregation comprising administering an amount up to 1500 mg/day of a compound ACI-3024 to a mammal subject in need thereof.

A method for preventing the formation of Tau aggregates and/or of inhibiting Tau aggregation comprising administering an amount up to 1500 mg/day of a compound ACI-3024 to a mammal subject in need.

A method for interfering intracellularly with Tau aggregates comprising administering an amount up to 1500 mg/day of a compound ACI-3024 to a mammal subject in need thereof.

A method for reducing Tau misfolding and hyperphosphorylation in vivo, the method comprising administering an amount up to 1500 mg/day of a compound ACI-3024 to a mammal subject in need thereof.

A method for reducing neuroinflammatory markers comprising administering an amount up to 1500 mg/day of a compound ACI-3024 to a mammal subject in need thereof.

A pharmaceutical composition comprising a compound ACI-3024 at an amount up to 1500 mg.

A pharmaceutical composition comprising a compound ACI-3024 and a Vitamin E Polyethylene Glycol Succinate (TPGS).

Any combination of the embodiments, preferred embodiments and more preferred embodiments disclosed herein is also envisaged in the present invention.

Any combination of the embodiments as disclosed in one aspect of the invention applies to the other aspects of the invention.

Following clauses are also part of the invention:
1. A compound ACI-3024 for use in the treatment, prevention or alleviation of a disorder or an abnormality associated with Tau protein aggregates or a tauopathy comprising administering said compound at an amount about 25 mg/day up to or equal to about 1500 mg/day to a mammal subject, preferably human subject, in need thereof.
2. The compound ACI-3024 for use according to clause 1 wherein the compound is given as single dose without inducing a serious adverse event in the subject.
3. The compound ACI-3024 for use according to clause 1 wherein the compound is given multiple times in a total amount below 1000 mg/day without inducing a serious adverse event in the subject.
4. The compound ACI-3024 for use according to clause 1 wherein ACI-3024 is administered at the amount up to about 1000 mg/day or about 500 mg/day.
5. A compound ACI-3024 for use in the treatment, prevention or alleviation of a disorder or an abnormality associated with Tau protein aggregates or a tauopathy comprising administering said compound at an amount from about 25 mg/day up to about 1500 mg/day to a human subject in need thereof wherein the compound ACI-3024 is characterized by a plasma half-life of at least 24 hours.
6. A compound ACI-3024 for use in the treatment, prevention or alleviation of a disorder or an abnormality associated with Tau protein aggregates or a tauopathy comprising administering to a mammal subject said compound at an amount that results in a Cmin concentration above or equal to 30 nM of said compound in CSF of the subject.
7. The compound ACI-3024 for use according to clause 1, 4 or 5 wherein the compound amount is up to or equal to about 300, 350, or 400 mg/day. Other embodiments may have the dose up to or equal to about 500, 550, 750, 1000 or 1400 mg/day.
8. The compound ACI-3024 for use according to clause 1, 4 or 5 wherein the compound amount is from about 300 or 400 mg/day.
9. The compound ACI-3024 for use according to clause 1, 4 or 5 wherein the compound is in the range of 300 mg/day to 350 mg/day.
10. The compound ACI-3024 for use according to clause 1 or any one of clause 3-9 wherein the daily dose is divided into unit dose(s) to be administered one, two or three times per day.
11. The compound ACI-3024 for use according to any one of the preceding clauses wherein the mammal subject is a human subject.
12. The compound ACI-3024 for use according to any one of the preceding clauses wherein ACI-3024 is administered orally.
13. The compound ACI-3024 for use according to clause 12 wherein said ACI-3024 is administered in the form of tablet, capsule, solution or suspension.
14. The compound ACI-3024 for use according to any one of the preceding clauses wherein the disorder or the abnormality associated with Tau protein aggregates or a tauopathy is selected from Alzheimer's disease (AD), familial AD, PART (Primary Age-Related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy including Lafora disease, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease.
15. The compound ACI-3024 for use according to clause 14 wherein the disorder or the abnormality associated with Tau protein aggregates or a tauopathy is selected from Alzheimer's disease (AD).
16. The compound ACI-3024 for use according to clause 14 wherein the disorder or the abnormality associated with Tau protein aggregates or a tauopathy is selected from progressive supranuclear palsy (PSP).
17. The compound ACI-3024 for use according to clause 14 wherein the disorder or the abnormality associated with Tau protein aggregates or a tauopathy is selected from frontotemporal lobar degeneration with predominant Tau pathology (FTLD-Tau).
18. The compound ACI-3024 for use according to clause 14 wherein the disorder or the abnormality associated with Tau protein aggregates or a tauopathy is selected from frontotemporal lobar degeneration caused by a MAPT gene mutation (FTLD-MAPT).
19. The compound ACI-3024 for use according to clause 14 wherein the disorder or the abnormality associated with Tau protein aggregates or a tauopathy is selected from corticobasal degeneration (CBD).
20. The compound ACI-3024 for use according to clause 14 wherein the disorder or the abnormality associated with Tau protein aggregates or a tauopathy is selected from Pick's disease (PiD).
21. The compound ACI-3024 for use according to clause 14 wherein the disorder or the abnormality associated with Tau protein aggregates or a tauopathy is Lafora Disease.
22. A method for treating, preventing or alleviating a disorder or an abnormality associated with Tau protein aggregates or a tauopathy by administering compound ACI-3024 at an amount from about 25 mg/day up to or equal to about 1500 mg/day to a mammal subject, preferably human subject, in need thereof.
23. The method according to clause 22 wherein the compound is given as single dose without inducing a serious adverse event in the subject.
24. A method for decreasing Tau aggregation comprising administering an amount of the compound ACI-3024 as defined in any one of clauses 1 to 10 to a mammal subject in need thereof.
25. A method for preventing the formation of Tau aggregates and/or of inhibiting Tau aggregation comprising administering an amount of the compound ACI-3024 as defined in any one of clauses 1 to 10 to a mammal subject in need thereof.
26. A method for interfering intracellularly with Tau aggregates comprising administering an amount of the compound ACI-3024 as defined in any one of clauses 1 to 10 to a mammal subject in need thereof.
27. A method for reducing Tau misfolding and hyperphosphorylation in vivo, the method comprising administering an amount of the compound ACI-3024 as defined in any one of clauses 1 to 10 to a mammal subject in need thereof.
28. A method for reducing neuroinflammatory markers comprising administering an amount of the compound ACI-3024 as defined in any one of clauses 1 to 10 to a mammal subject in need thereof.
29. A pharmaceutical composition comprising compound ACI-3024 in an amount as defined in any one of clauses 1 to 10.
30. The pharmaceutical composition according to clause 29 comprising additionally a Vitamin E Polyethylene Glycol Succinate (TPGS) to be administered orally to a subject in need.
31. A method for obtaining the pharmaceutical composition as defined in clause 30, wherein a solution comprising 3% TPGS/water is mixed with a solid form of ACI-3024 for obtaining a suspension.

Following clauses are also part of the invention:
1. A compound ACI-3024 for use in the treatment, prevention or alleviation of a disorder or an abnormality associated with Tau protein aggregates comprising administering said compound at an amount up to 1500 mg/dose/day to a mammal subject in need thereof without inducing a severe adverse event in the subject when administered as a single dose.
2. A compound ACI-3024 for use in the treatment, prevention or alleviation of a disorder or an abnormality associated with Tau protein aggregates comprising administering said compound in one or more of the dose(s) described herein to a mammal subject in need thereof without inducing a severe adverse event in the subject 3. The compound according to clause 1 wherein ACI-3024 is administered at the amount of about 25 mg/dose/day to 1500 mg/dose/day.
4. The compound according to clause 2 wherein the amount is selected from 25, 100, 300, 500, 550, 750, 1000, 1400 and 1500 mg/dose/day.
5. The compound according to any preceding clauses wherein the daily dose is divided into unit dose(s) to be administered one, two or three times per day.
6. The compound according to any preceding clauses wherein the mammal subject is a human subject.
7. The compound according to any of preceding clauses wherein ACI-3024 is administered orally.
8. The compound according to clause 6 wherein said ACI-3024 is administered in the form of tablet, capsule, solution or suspension.
9. The compound according to any of preceding clauses wherein the disorder or the abnormality associated with Tau protein aggregates is selected from Alzheimer's disease (AD), familial AD, PART (Primary Age-Related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease.
10. The compound according to clause 8 wherein the disorder or the abnormality associated with Tau protein aggregates is selected from Alzheimer's disease (AD) and progressive supranuclear palsy (PSP).
11. A method for treating, preventing or alleviating a disorder or an abnormality associated with Tau protein aggregates by administering the compound ACI-3024 at an amount up to 1500 mg/dose/day to a mammal subject in need thereof without inducing a severe adverse event in the subject.
12. The method according to clause 10 wherein ACI-3024 is administered at the amount of about 25 mg/dose/day to 1500 mg/dose/day.
13. The method according to clause 11 wherein the amount is selected from 25, 100, 300, 500, 550, 750, 1000, 1400 and 1500 mg/dose/day.
14. The method according to clauses 10, 11 or 12 wherein the daily dose is divided into unit dose(s) to be administered one, two or three times per day.
15. The method according to clauses 10, 11, 12 or 13 wherein the mammal subject is a human subject.
16. The method according to clauses 10, 11, 12, 13 or 14 wherein ACI-3024 is administered orally.
17. The compound according to clause 15 wherein said ACI-3024 is administered in the form of tablet, capsule, solution or suspension.
18. The method according to clauses 10 to 16 wherein the disorder or the abnormality associated with Tau protein aggregates is selected from Alzheimer's disease (AD), familial AD, PART (Primary Age-Related Tauopathy), Creutzfeldt-Jacob disease, dementia pugilistica, Down's Syndrome, Gerstmann-Sträussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease.
19. The method according to clause 17 wherein the disorder or the abnormality associated with Tau protein aggregates is selected from Alzheimer's disease (AD) and progressive supranuclear palsy (PSP).
20. A method for decreasing Tau aggregation comprising administering an amount of the compound ACI-3024 as defined in any one of clauses 1 to 7 to a mammal subject in need thereof without inducing a severe adverse event in the subject.
21. A method for preventing the formation of Tau aggregates and/or of inhibiting Tau aggregation comprising administering an amount of the compound ACI-3024 as defined in any one of clauses 1 to 7 to a mammal subject in need thereof without inducing a severe adverse event in the subject.
22. A method for interfering intracellularly with Tau aggregates comprising administering an amount of the compound ACI-3024 as defined in any one of clauses 1 to 7 to a mammal subject in need thereof without inducing a severe adverse event in the subject.
22. A method for reducing Tau misfolding and hyperphosphorylation in vivo, the method comprising administering an amount of the compound ACI-3024 as defined in any one of clauses 1 to 7 to a mammal subject in need thereof without inducing a severe adverse event in the subject.
23. A method for reducing neuroinflammatory markers comprising administering an amount of the compound ACI-3024 as defined in any one of clauses 1 to 7 to a mammal subject in need thereof without inducing a severe adverse event in the subject.
24. A pharmaceutical composition comprising ACI-3024 at an amount up to 1500 mg.
25. The pharmaceutical composition according to clause 24 comprising additionally a Vitamin E Polyethylene Glycol Succinate (TPGS) to be administered orally to a subject in need.
26. A method for obtaining the pharmaceutical composition as defined in clause 25, wherein a solution comprising 3% TPGS/water is mixed with a solid form of ACI-3024 for obtaining a suspension.

### BRIEF DESCRIPTION OF THE FIGURES

FIG. 1: Mean plasma concentrations of ACI-3024 following single oral doses in healthy volunteers.

FIG. 2: ACI-3024 plasma exposure (AUC0-tlast) versus dose following single oral doses in healthy volunteers.

FIG. 3: ACI-3024 CSF concentrations in healthy volunteers following multiple oral doses of 1400 mg at Day 7 (Cmin=Predose) and on day 14, (Cmax=4 to 8 hours Postdose).

### DETAILED DESCRIPTION OF THE INVENTION

The use of compound ACI-3024 and method related to compound ACI-3024 are within the scope of the present invention and will be described in the following. Further, the invention is directed to a pharmaceutical composition comprising a compound ACI-3024 as described in the present invention. It is to be understood that all possible combinations of the following embodiments are also envisaged.

Treatment Dosage:

The present invention is directed to a compound ACI-3024 for use in the treatment, prevention or alleviation of a disorder or an abnormality associated with Tau protein aggregates or a tauopathy comprising administering said compound at an amount up to or equal to about 1500 (one thousand five hundred) mg/day to a mammal subject in need thereof, preferably a human subject.

In other words, the present invention is directed to a method for treatment, prevention or alleviation of a disorder or an abnormality associated with Tau protein aggregates or a tauopathy comprising administering compound ACI-3024 at an amount up to or equal to about 1500 mg/day to a mammal subject in need thereof, preferably a human subject.

In one embodiment, the present invention is directed to a method for treatment, prevention or alleviation of a disorder or an abnormality associated with Tau protein aggregates or a tauopathy comprising administering compound ACI-3024 at an amount at about 25 mg/day to about 1500 mg/day to a mammal subject in need thereof, preferably a human subject.

In one embodiment, the compound ACI-3024 for use in the treatment, prevention or alleviation of a disorder or an abnormality associated with Tau protein aggregates or a tauopathy comprises administering said compound in an amount from about 25 mg/day up to or equal to about 1500 mg/day to a mammal subject in need thereof, preferably a human subject.

In one embodiment, the compound ACI-3024 for use in the treatment, prevention or alleviation of a disorder or an abnormality associated with Tau protein aggregates or a tauopathy comprises administering said compound from an amount about 175 mg/day up to or equal to about 1500 mg/day to a mammal subject in need thereof, preferably a human subject.

Preferably, said compound is administered at an amount from about 350 mg/day to about 500 mg/day.

The present invention is directed to a safe use of compound ACI-3024 in the treatment, prevention or alleviation of a disorder or an abnormality associated with Tau protein aggregates or a tauopathy comprising administering said compound at an amount up to 1500 mg/day to a mammal subject, preferably human subject, in need thereof.

In other words, the invention is directed to a compound ACI-3024 for the safe use in the treatment, prevention or alleviation of a disorder or an abnormality associated with Tau protein aggregates comprising administering said compound at an amount up to 1500 mg/day to a mammal subject in need thereof without inducing.

In one embodiment, the compound ACI-3024 for use in the treatment, prevention or alleviation of a disorder or an abnormality associated with Tau protein aggregates or a tauopathy comprises administering said compound as disclosed herein where the compound is given as single dose without inducing a serious adverse event in the subject.

ACI-3024 Half-Life in Plasma

The present invention is directed to a compound ACI-3024 for use in the treatment, prevention or alleviation of a disorder or an abnormality associated with Tau protein aggregates or a tauopathy comprising administering said compound at an amount up to or equal to about 1500 mg/day to a human subject in need thereof wherein the compound ACI-3024 is characterized by a plasma half-life of at least 24 hours.

In other words, the present invention is directed to a method for treatment, prevention or alleviation of a disorder or an abnormality associated with Tau protein aggregates or a tauopathy comprising administering compound ACI-3024 at an amount up to or equal to about 1500 mg/day to a human subject in need thereof wherein the compound ACI-3024 is characterized by a plasma half-life of at least 24 hours.

In one embodiment, the compound ACI-3024 for use in the treatment, prevention or alleviation of disorder or abnormality associated with Tau protein aggregates or a tauopathy comprises administering said compound at an amount about 25 mg/day up to or equal to about 1500 mg/day to a human subject in need thereof, wherein the compound ACI-3024 is characterized by a plasma half-life of at least 24 hours.

In one embodiment, the compound ACI-3024 for use in the treatment, prevention or alleviation of disorder or abnormality associated with Tau protein aggregates or a tauopathy comprises administering said compound at an amount from about 350 mg/day up to or equal to about 500 mg/day to a human subject in need thereof, wherein the compound ACI-3024 is characterized by a plasma half-life of at least 24 hours.

The half-life is of at least 24 hours for administered dose of said compound at an amount up to or equal to 1500 mg/day. Preferably, the half-life is in the range of 24 hours to 120 hours.

Cerebrospinal Fluid (CSF) Concentration:

The present invention is directed to a compound ACI-3024 for use in the treatment, prevention or alleviation of a disorder or an abnormality associated with Tau protein aggregates or a tauopathy comprising administering said compound at an amount up to or equal to 1500 mg/day to a human subject in need thereof wherein the compound ACI-3024 is constantly present in the human subject CSF at a concentration above or equal to 30 nM level at predose, preferably above or equal to 90 nM level at predose. The predefined predose level of ACI-3024 of 30 nM is derived from studies of ACI-3024 in the Tg4510 transgenic mouse model which reflects the clinical manifestation of tauopathies and determines the minimum concentration of the compound in the CSF necessary to achieve clinical efficacy before another dose of the same compound is given (ie a threshold value below which efficacy would be lost). Preferably, the compound ACI-3024 is present in the human subject CSF at a concentration above or equal to 30 nM, preferably above or equal to 90 nM, during the treatment.

In other words, the present invention is directed to a method for treatment, prevention or alleviation of a disorder or an abnormality associated with Tau protein aggregates or a tauopathy comprising administering compound ACI-3024 at an amount up to or equal to 1500 mg/day to a human subject in need thereof wherein the compound ACI-3024 is constantly or consistently present in the CSF of a human subject at a concentration above or equal to 30 nM, preferably above or equal to 90 nM. If and/or when the concentration of ACI-3024 is measured or predicted to drop below 30 nM (and more preferably below 90 nM), one or more additional doses of the compound may be administered to ensure that the desired amount is constantly or consistently present in the patient.

Methods for measuring the amount of administered compound, such as ACI-3024, in CSF of a subject are well know in the art. As an example, CSF can be drawn by lumbar puncture and content of the administered compound can be analysed by a LC-MS/MS method, among other methods.

The embodiments and preferred descriptions are applying to the described above inventions and can be combined with each others.

The compound ACI-3024 is defined in WO2019/134978A1 having the following structure:

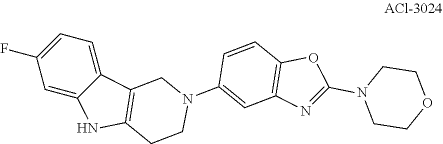 [SMILES: Fc1ccc2c3c([nH]c2c1)CCN(c1ccc2oc(N4CCOCC4)nc2c1)C3]

Compound ACI-3024 means any free base, tautomers, pharmaceutically acceptable salts (that may include stereoisomers or racemic mixtures thereof), hydrates, or solvates of ACI-3024, indicated as ACI-3024 herewith.

In one embodiment, ACI-3024 is preferably a free base or tautomer of ACI-3024. More preferably, ACI-3024 is a free base.

In a further embodiment, ACI-3024 is preferably a pharmaceutically acceptable salt (that may include stereoisomers or racemic mixtures thereof), hydrate, or solvate of ACI-3024. In one embodiment, ACI-3024 is administered at an amount in the range of 25 mg/day to 1500 mg/day; 100 mg/day to 1500 mg/day; 175 mg/day to 1500 mg/day; 300 mg/day to 1500 mg/day; 350 mg/day to 1500 mg/day; 500 mg/day to 1500 mg/day; 550 mg/day to 1500 mg/day; 550 mg/day to 1400 mg/day; 750 mg/day to 1500 mg/day; 750 mg/day to 1500 mg/day; 350 mg/day to 500 mg/day. Preferably, ACI-3024 is administered at an amount in the range of 300 mg/day to 1500 mg/day, 350 m/day to 500 mg/day; 550 mg/day to 1400 mg/day or 500 mg/day to 1500 mg/day.

In a further embodiment, ACI-3024 is administered at an amount of 25, 100, 300, 350, 400, 450, 500, 550, 750, 1000, 1400 or 1500 mg/day. Preferably, ACI-3024 is administered at an amount of 300, 350, 400, 450, 500, 550, 750, 1000, 1400 or 1500 mg/day. Even more preferably, ACI-3024 is administered at an amount of 350, 400, 450, 500, 550, 750, 1000, 1400 or 1500 mg/day.

In a further embodiment, ACI-3024 is administered at an amount up to 1400, 1000, 900, 800, 700, 600, 550, 500, 450, 400, 350 or 300 mg/day. Preferably, ACI-3024 is administered at an amount up to 1000, 900, 800, 700, 600, 550, 500, 450, 400, 350 or 300 mgday. Even more preferably, ACI-3024 is administered at an amount up to 900, 800, 700, 600, 550, 500, 450, 400, 350 or 300 mg/day. Even more preferably, ACI-3024 is administered at an amount up to 800, 700, 600, 550, 500, 450, 400, 350 or 300 mg/day. Even more preferably, ACI-3024 is administered at an amount up to 700, 600, 550, 500, 450, 400, 350 or 300 mg/day. Even more preferably, ACI-3024 is administered at an amount up to 600, 550, 500, 450, 400, 350 or 300 mg/day. Even more preferably, ACI-3024 is administered at an amount up to 500, 450, 400, 350 or 300 mg/day. Even more preferably, ACI-3024 is administered at an amount up to 500, 450, 400, or 350 mg/day.

In a further embodiment, ACI-3024 is administered at an amount in the range of 25 mg/day to 1000 mg/day; 100 mg/day to 1000 mg/day; 100 mg/day to 900 mg/day; 100 mg/day to 750 mg/day; 200 mg/day to 750 mg/day; 300 mg/day to 750 mg/day; 300 mg/day to 600 mg/day; 300 mg/day to 500 mg/day or 350 mg/day to 500 mg/day. Preferably, ACI-3024 is administered at an amount in the range of 100 mg/day to 1000 mg/day; 100 mg/day to 900 mg/day; 100 mg/day to 750 mg/day; 200 mg/day to 750 mg/day; 300 mg/day to 750 mg/day; 300 mg/day to 600 mg/day; 300 mg/day to 500 mg/day or 350 mg/day to 500 mg/day. Even more preferably, ACI-3024 is administered at an amount in the range of 200 mg/day to 750 mg/day; 300 mg/day to 750 mg/day; 300 mg/day to 600 mg/day; 300 mg/day to 500 mg/day; 350 mg/day to 500 mg/day. Even more preferably, ACI-3024 is administered at an amount in the range of 350 mg/day to 500 mg/day.

In one embodiment, ACI-3024 is administered a daily dose that is optionally divided into unit dose(s) to be administered one, two or three times per day. The daily dose is the total dose to be administered to subject in need wherein the total dose can be divided in unit dose(s) administered along the day (no more than 24 hours).

In one embodiment, ACI-3024 is administered to a mammal subject wherein the subject is preferably a human. In one embodiment, ACI-3024 is administered to a subpopulation consisting of human beings that are first-generation Japanese. As used herein, “first-generation Japanese” means that the subject, the subject's biological parents, and all the subject's biological grandparents are of exclusively Japanese descent and were all born in Japan.

In one embodiment, ACI-3024 is administered preferably in the form of a tablet, capsule, solution or suspension. More preferably, ACI-3024 is administered orally in the form of a solution or suspension. More preferably, ACI-3024 is administered orally in the form of a tablet or capsule.

In one embodiment, the use of ACI-3024 is for treatment, prevention or alleviation of a disorder or an abnormality associated with Tau protein aggregates or a tauopathy wherein the disorder or the abnormality associated with Tau protein aggregates or a tauopathy is selected from Alzheimer's disease (AD), familial AD, PART (Primary Age-Related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy including Lafora disease, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease.

Preferably, the diseases or the abnormality associated with Tau protein aggregates, is selected from Alzheimer's disease (AD), as well as other neurodegenerative tauopathies such as Creutzfeldt-Jacob disease, dementia pugilistica, amyotrophic lateral sclerosis (ALS), argyrophilic grain disease (AGD), corticobasal degeneration (CBD), frontotemporal dementia with Parkinsonism linked to chromosome 17 (FTDP-17), Pick's disease (PiD), progressive supranuclear palsy (PSP), tangle predominant dementia, Parkinson dementia complex of Guam, Hallervorden-Spatz disease, chronic traumatic encephalopathy (CTE), traumatic brain injury (TBI), and other frontotemporal lobar degeneration.

More preferably, the disorder or the abnormality associated with Tau protein aggregates or a tauopathy is selected from Alzheimer's disease (AD), corticobasal degeneration (CBD), Pick's disease (PiD), progressive supranuclear palsy (PSP), and frontotemporal lobar degeneration with predominant Tau pathology (FTLD-Tau) including frontotemporal lobar degeneration caused by a MAPT gene mutation (FTLD-MAPT).

Even more preferably, the disorder or the abnormality associated with Tau protein aggregates is selected from Alzheimer's disease (AD) and progressive supranuclear palsy (PSP).

In one embodiment, the disorder or the abnormality associated with Tau protein aggregates or a tauopathy is selected from Alzheimer's disease (AD).

In one embodiment, the disorder or the abnormality associated with Tau protein aggregates or a tauopathy is selected from progressive supranuclear palsy (PSP).

In one embodiment, the disorder or the abnormality associated with Tau protein aggregates or a tauopathy is selected from frontotemporal lobar degeneration with predominant Tau pathology (FTLD-Tau) including frontotemporal lobar degeneration caused by a MAPT gene mutation (FTLD-MAPT).

In one embodiment, the disorder or the abnormality associated with Tau protein aggregates or a tauopathy is selected from corticobasal degeneration (CBD).

In one embodiment, the disorder or the abnormality associated with Tau protein aggregates or a tauopathy is selected from Pick's disease (PiD).

The present invention is further directed to a method for treating, preventing or alleviating a disorder or an abnormality associated with Tau protein aggregates by administering ACI-3024 at an amount up to 1500 mg/day to a mammal subject in need thereof.

In other words, the invention is directed to a method for treating, preventing or alleviating a disorder or an abnormality associated with Tau protein aggregates comprising the step of administering ACI-3024 at an amount up to 1500 mg/day to a mammal subject in need thereof.

In one embodiment, the method comprises the step of administering ACI-3024 that is preferably a free base or tautomer of ACI-3024. More preferably, ACI-3024 is a free base.

In a further embodiment, the method comprises the step of administering ACI-3024 that is preferably a pharmaceutically acceptable salt (that may include stereoisomers or racemic mixtures thereof), a hydrate, or a solvate of ACI-3024.

In one embodiment, ACI-3024 is preferably a free base or tautomer of ACI-3024. More preferably, ACI-3024 is a free base.

In one embodiment, the method for treatment, prevention or alleviation of a disorder or an abnormality associated with Tau protein aggregates or a tauopathy comprises administering said compound as disclosed herein where the compound is given as single dose without inducing a serious adverse event in the subject.

In a further embodiment, ACI-3024 is preferably a pharmaceutically acceptable salt (that may include stereoisomers or racemic mixtures thereof), hydrate, or solvate of ACI-3024.

In one embodiment, the method comprises the step of administering ACI-3024 at an amount in the range of 25 mg/day to 1500 mg/day; 300 mg/day to 1500 mg/day; 500 mg/day to 1500 mg/day; 550 mg/day to 1500 mg/day; 550 mg/day to 1400 mg/day; 750 mg/day to 1400 mg/day; 750 mg/day to 1500 mg/day. Preferably, the method comprises the step of administering ACI-3024 at an amount in the range of 300 mg/day to 1500 mg/day, 550 mg/day to 1400 mg/day or 500 mg/day to 1500, or 350 mg/day to 500 mg/day.

In a further embodiment, the method comprises the step of administering ACI-3024 at an amount of 25, 100, 300, 350, 500, 550, 750, 1000, 1400 or 1500 mg/day. Preferably, the method comprises the step of administering ACI-3024 at an amount of 300, 350, 500, 550, 750, 1000, 1400 or 1500 mg/day. Even more preferably, the method comprises the step of administering ACI-3024 at an amount of 350, 500, 550, 750, 1000, 1400 or 1500 mg/day.

In one embodiment, the method comprises the step of administering ACI-3024 with a daily dose that is optionally divided into unit dose(s) to be administered one, two or three times per day. The daily dose is the total dose to be administered to a subject in need thereof wherein the total dose can be divided in unit dose(s) administered along the day (no more than 24 hours).

In one embodiment, the method comprises the step of administering ACI-3024 that is administered to a mammal subject wherein the subject is preferably a human. In one embodiment, ACI-3024 is administered to a subpopoulation consisting of human beings that are first-generation Japanese. As used herein, “first-generation Japanese” means that the subject, the subject's biological parents, and all the subject's biological grandparents are of exclusively Japanese descent and were born in Japan.

In one embodiment, the method comprises the step of administering ACI-3024 is administered preferably orally in the form of a tablet, capsule, solution or suspension. More preferably, ACI-3024 is administered orally in the form of a solution or suspension. More preferably, ACI-3024 is administered orally in the form of a tablet or capsule.

In one embodiment, the method comprises the step of administering ACI-3024 is for treatment, prevention or alleviation of a disorder or an abnormality associated with Tau protein aggregates wherein the disorder or the abnormality associated with Tau protein aggregates is selected from Alzheimer's disease (AD), familial AD, PART (Primary Age-Related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis (SSPE), tangle predominant dementia, postencephalitic Parkinsonism, myotonic dystrophy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy including Lafora disease, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease.

Preferably, the diseases or the disorder or the abnormality associated with Tau protein aggregates, is selected from Alzheimer's disease (AD), as well as other neurodegenerative tauopathies such as Creutzfeldt-Jacob disease, dementia pugilistica, amyotrophic lateral sclerosis (ALS), argyrophilic grain disease (AGD), corticobasal degeneration (CBD), frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Pick's disease (PiD), progressive supranuclear palsy (PSP), tangle predominant dementia, Parkinson dementia complex of Guam, Hallervorden-Spatz disease, chronic traumatic encephalopathy (CTE), traumatic brain injury (TBI), and other frontotemporal lobar degeneration.

More preferably, the diseases or the disorder or the abnormality associated with Tau protein aggregates is selected from Alzheimer's disease (AD), corticobasal degeneration (CBD), Pick's disease (PiD), progressive supranuclear palsy (PSP), FTLD-MAPT (frontotemporal lobar degeneration caused by a MAPT gene mutation), and frontotemporal lobar degeneration with predominant Tau pathology (FTLD-Tau).

Even more preferably, the disease or the disorder or the abnormality associated with Tau protein aggregates is selected from Alzheimer's disease (AD), progressive supranuclear palsy (PSP), FTLD-MAPT (frontotemporal lobar degeneration caused by a MAPT gene mutation) and frontotemporal lobar degeneration with predominant Tau pathology (FTLD-Tau).

The present invention is further directed to a method for decreasing Tau aggregation comprising administering an amount of ACI-3024 as defined above to a mammal subject in need thereof.

The present invention is further directed to a method for preventing the formation of Tau aggregates and/or of inhibiting Tau aggregation comprising administering an amount of ACI-3024 as defined above to a mammal subject in need thereof.

The present invention is further directed to a method for interfering intracellularly with Tau aggregates comprising administering an amount of ACI-3024 as defined above to a mammal subject in need thereof.

The present invention is further directed to a method for reducing Tau misfolding and hyperphosphorylation in vivo, the method comprising administering an amount of ACI-3024 as defined above to a mammal subject in need thereof.

The present invention is further directed to a method for reducing neuroinflammatory markers comprising administering an amount of ACI-3024 as defined above to a mammal subject in need thereof.

The present invention is further directed to a method for treating, preventing or alleviating a disorder or an abnormality associated with Tau protein aggregates by administering ACI-3024 to a mammal subject in need thereof, wherein the maximal exposure (AUC₀₋₂₄ₕ) of the subject is not higher than 360 ug*h/ml. In one embodiment, the maximal exposure (AUC₀₋₂₄ₕ) of the subject is preferably 353 ug*h/ml.

The present invention is further directed to a method for treating, preventing or alleviating a disorder or an abnormality associated with Tau protein aggregates by administering ACI-3024 to a mammal subject in need thereof without inducing a serious adverse event in said subject wherein ACI-3024 is administered under Fasting or Fed Conditions. Preferably, the daily dose is administered under Fasting and/or Fed Conditions whereas daily dose is divided into unit dose(s) administered under Fed Condition and unit dose(s) administered under Fasting Condition. More preferably, a first unit dose of ACI-3024 is administered under Fasting Condition and other unit dose(s) are administered under Fed Condition.

The present invention is further directed to a pharmaceutical composition comprising ACI-3024 at an amount up to 1500 mg. The pharmaceutical composition comprises additionally Vitamin E Polyethylene Glycol Succinate (TPGS) to be administered orally to a subject in need.

In one embodiment, the pharmaceutical composition comprises ACI-3024 at an amount up to 1500 mg for use in the treatment, prevention or alleviation of a disorder or an abnormality associated with Tau protein aggregates to be administered to a mammal subject in need thereof.

In one embodiment, the pharmaceutical composition is comprising ACI-3024 at an amount up to 1500 mg and Vitamin E Polyethylene Glycol Succinate (TPGS). Preferably, the pharmaceutical composition is administered orally in the form of tablet, capsule, solution or suspension to a subject in need. More preferably, the pharmaceutical composition is administered orally in the form of capsule, to a subject in need. More preferably, the pharmaceutical composition is administered orally in the form of solution or suspension to a subject in need.

In one embodiment, the pharmaceutical composition is obtained by dissolving TPGS in water to obtain a solution of 3% TPGS/water and then adding a solid form of ACI-3024 at an amount up to 1500 mg for obtaining a suspension.

In other words, the method for obtaining the pharmaceutical composition comprises the step of mixing a solution comprising 3% TPGS/water with a solid form of ACI-3024 at an amount up to 1500 mg for obtaining a suspension.

Any combination of the embodiments, preferred embodiments and more preferred embodiments disclosed herein is also envisaged in the present invention. The disclosed herewith embodiments can be combined with each other.

Pharmaceutical Compositions

While it is possible for the compound of the present invention to be administered alone, it is preferable to formulate them into a pharmaceutical composition in accordance with standard pharmaceutical practice. Thus, the invention also provides a pharmaceutical composition which comprises a therapeutically effective amount of a compound ACI-3024 optionally in admixture with a pharmaceutically acceptable carrier, diluent, adjuvant or excipient.

Pharmaceutically acceptable excipients are well known in the pharmaceutical art, and are described, for example, in Remington's Pharmaceutical Sciences, 15^{th} Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

Pharmaceutically useful excipients that may be used in the formulation of the pharmaceutical composition of the present invention may comprise, for example, carriers, vehicles, diluents, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone, low melting waxes, and ion exchange resins.

The routes for administration (delivery) of the compounds of the invention include, but are not limited to, one or more of: oral (e. g. as a tablet, capsule, solution, suspension or as an ingestible solution), topical, mucosal (e. g. as a nasal spray or aerosol for inhalation), nasal, parenteral (e. g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

For example, the compound can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

If the compound of the present invention are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compound of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e. g. using a mixture of ethanol and the propellant as the solvent, which may additionally contain a lubricant, e. g. sorbitan trioleate. Capsules and cartridges (made, for example, from gelatin) for use in an inhaler or insufflator may be formulated to contain a powder mix of the compound and a suitable powder base such as lactose or starch.

Alternatively, the compound of the present invention can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH was adjusted, sterile saline, or, preferably, as solutions in isotonic, pH was adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The compound of the invention may also be used in combination with other therapeutic agents. When a compound of the invention is used in combination with at least one therapeutic agent active against the same disease, the dose of each compound may differ from that when the compound is used alone.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the second therapeutic agent may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manner as are known for such compounds in the art.

The pharmaceutical compositions of the invention can be produced in a manner known per se to the skilled person as described, for example, in Remington's Pharmaceutical Sciences, 15^{th} Ed., Mack Publishing Co., New Jersey (1975).

The diseases or conditions that can be treated, alleviated or prevented with the compounds of the present invention are disorders or abnormalities associated with Tau protein aggregates (i.e. tauopathies) such as neurodegenerative disorders. Examples of diseases and conditions which can be treated, alleviated or prevented are caused by or associated with the formation of neurofibrillary lesions. This is the predominant brain pathology in tauopathy. The diseases and conditions comprise a heterogeneous group of neurodegenerative diseases or conditions including diseases or conditions which show co-existence of Tau and amyloid pathologies.

Examples of the diseases and conditions which can be treated, alleviated or prevented include, but are not limited, to Alzheimer's disease (AD), familial AD, PART (Primary Age-Related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy including Lafora disease, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease. Preferably the diseases and conditions which can be treated, alleviated or prevented include Alzheimer's disease (AD), as well as other neurodegenerative tauopathies such as Creutzfeldt-Jacob disease, dementia pugilistica, amyotrophic lateral sclerosis (ALS), argyrophilic grain disease (AGD), corticobasal degeneration (CBD), frontotemporal dementia with Parkinsonism linked to chromosome 17 (FTDP-17), Pick's disease (PiD), progressive supranuclear palsy (PSP), tangle predominant dementia, Parkinson dementia complex of Guam, Hallervorden-Spatz disease, chronic traumatic encephalopathy (CTE), traumatic brain injury (TBI), and other frontotemporal lobar degeneration. More preferably Alzheimer's disease (AD), corticobasal degeneration (CBD), Pick's disease (PiD), progressive supranuclear palsy (PSP) FTLD-MAPT (frontotemporal lobar degeneration caused by a MAPT gene mutation) and frontotemporal lobar degeneration with predominant Tau pathology (FTLD-Tau).

The use of the compound ACI-3024 of the present invention with disclosed daily doses can also be employed to decrease protein aggregation, in particular Tau aggregation in mammal, preferably human. The ability of a compound to decrease of Tau aggregation can, for example, be determined using the ThT assay (Hudson et al., FEBS J., 2009, 5960-72).

The compound of the invention can be used safely in the treatment of a wide range of disorders in which the neuroinflammation process is associated with misfolding and/or aggregation of Tau protein.

### Definitions

The term “Adverse Event” (AE) means any untoward medical occurrence in a subject or clinical investigation subject administered with a pharmaceutical product (study drug), which does not necessarily have a causal relationship with this treatment. An AE can, therefore, be any unfavorable and/or unintended sign (including a clinically significant abnormal laboratory finding), symptom, or disease temporally associated with the use of a study drug, whether or not related to the study drug.

The term “Serious Adverse Event” means an AE occurring at any dose that results in any of the following outcomes: Death; life-threatening (refers to an event in which the subject was at risk of death at the time of the event; it does not refer to an event which could hypothetically have caused death had it been more severe); inpatient hospitalization or prolongation of an existing hospitalization (refers to an unplanned, overnight hospitalization); Persistent or significant disability/incapacity; congenital anomaly/birth defect; important medical event (an event not meeting the above criteria but deemed by the investigator) to be one that may jeopardize the subject or may require intervention to prevent one of the other outcomes listed above (e.g. intensive treatment in an emergency room or at home for allergic bronchospasm or blood dyscrasias or convulsions that do not result in hospitalization).

The term severe adverse event is used to describe the intensity (severity) of a adverse event (as in mild, moderate, or severe); the event itself, however, may be of relatively minor medical significance (such as severe headache).

The term “Cmin” is the minimum (or trough) serum concentration that a drug achieves in a specified compartment or test area of the body after the drug has been administered and before the administration of a second dose. Cmin is the opposite of Cmax, which is the maximum (or peak) concentration that a drug achieves after dosing.

### Example 1

A First-In-Human, Randomized, Placebo-Controlled, Double-Blind, SAD/MAD Study to Assess the Safety, Tolerability, Pharmacokinetics, and Pharmacodynamics of ACI-3024

Study Objective:

The objective of the study is to assess the safety, tolerability, pharmacokinetics (PK), and pharmacodynamics (PD) of single ascending doses (SAD) and multiple ascending doses (MAD) of ACI-3024 in a first-in-human, randomized, placebo-controlled, double-blind, sequential SAD/MAD study with open-label food effect and pharmacodynamic assessment arms. The study comprises different parts to be conducted in Healthy Volunteers:
1) one Single Ascending Dose (SAD) part in which several cohorts are being investigated with single ascending doses of ACI-3024. The food effect is also explored in this part.
2) one Multiple Ascending Dose (MAD) part in which several cohorts are being investigated with multiple ascending doses of ACI-3024. Each study part is assessing the safety, tolerability, pharmacokinetics and pharmacodynamics of ACI-3024.

Primary Objectives:
To assess the safety and tolerability of ACI-3024 in study participants following single and multiple oral doses.

Secondary Objectives:
To assess the PK of ACI-3024 in plasma, urine, and cerebrospinal fluid (CSF).
To compare the plasma and urine PK parameters of ACI-3024 following single oral doses in the fed and fasting states.

Exploratory Objectives:
To explore the effect of ACI-3024 on putative biomarkers of AD, including concentrations of total Tau, p-Tau, neurogranin, and neurofilament light chain, in blood and/or CSF.
To explore the effect of sex and age on the safety, tolerability, and PK parameters of ACI-3024.
To optionally explore the metabolite profile of ACI-3024 in plasma, CSF, and urine.
To explore the safety, tolerability, PK, and biomarkers of ACI-3024 in Japanese subjects in relation to non-Japanese subjects.

Inclusion Criteria:
Healthy subjects must satisfy all of the following criteria at the Screening visit unless otherwise stated:
   Males (or females, when applicable) either 20 to 59 years old, inclusive (non-elderly subjects), or 60 to 80 years old, inclusive (elderly subjects), depending on the cohort to which they are recruited:
   Cohort S1: Non-elderly healthy males
   Cohorts S2 to S5: Non-Japanese (preferred to be non-Asian) and Japanese non-elderly healthy males
   Cohort MAD-HV1 to MAD-HV3: Elderly males or females
   Cohort MAD-HV4: Non-Japanese (preferred to be non-Asian) and Japanese non-elderly males.
Japanese subjects must be first-generation Japanese, defined as the subject, the subject's biological parents, and all the subject's biological grandparents are of exclusively Japanese descent and were born in Japan. Non-Japanese subjects are preferred to be non-Asian.
For non-Japanese subjects: Body mass index between 18.0 and 30.0 kg/m2, inclusive, and body weight between 50 and 100 kg (males) or between 45 and 80 kg (females), inclusive. For Japanese subjects: Body mass index between 17.0 and 30.0 kg/m2 and body weight between 45 and 100 kg (males) or between 40 and 80 kg (females), inclusive.
In good health, determined by no clinically significant findings from medical history, physical examination, 12 lead ECG, vital signs measurements, and clinical laboratory evaluations (congenital nonhemolytic hyperbilirubinemia [eg, suspicion of Gilbert's syndrome based on total and direct bilirubin] or any clinical and/or laboratory condition anticipated to affect ACI-3024 binding to plasma proteins is not acceptable) at Screening and Check in as assessed by the Investigator (or designee).
Systolic blood pressure ≥90 mmHg and ≤150 mmHg (elderly subjects) or ≥90 mmHg and ≥140 mmHg (non-elderly subjects), diastolic blood pressure ≥50 mmHg and ≤90 mmHg, and resting pulse rate ≥45 bpm and ≤100 bpm.
Have not used tobacco or nicotine containing products within 3 months prior to Check in, and has a negative cotinine test at Screening and Check-in.
Females must not be pregnant or lactating, and females of childbearing potential and males will agree to use contraception.
Able to comprehend and willing to sign an ICF and to abide by the study restrictions.
For subjects in Cohort MAD-HV3, willing to undergo three lumbar punctures and associated CSF sampling.

The study contains different parts:

Single Ascending Dose (SAD) Part:

This part comprised a single dose, sequential group design. Overall, 40 healthy non-elderly male subjects were enrolled in 5 cohorts (Cohorts S1 to S5), with each cohort consisting of 8 subjects.

No Japanese subjects were enrolled in Cohort S1. For Cohorts S2 through S5, 4 Japanese subjects and 4 non Japanese subjects were enrolled.

In each of Cohorts S1 to S5, 6 subjects (including 3 Japanese subjects in Cohorts S2 through S5) received ACI-3024 and 2 subjects (including 1 Japanese subject in Cohorts S2 through S5) received placebo. All doses were administered in the fasting state in accordance with a randomization schedule on the morning of Day 1, except for subjects in the food-effect cohort (Cohort S4), who were dosed 30 minutes after starting a high-fat breakfast in Treatment Period 2 only. Each subject received only a single dose of ACI-3024 or placebo during the study, except for subjects in the food effect cohort, who received a single dose of ACI-3024 or placebo in the fasting state followed by a single dose of ACI-3024 in the fed state. Thus, subjects in Cohort S4 received either 2 identical doses of ACI-3024 (6 subjects) or 1 dose of placebo and 1 dose of ACI-3024 (2 subjects). Six subjects of Cohort S4 received 750 mg dose of ACI-3024 and 2 subjects received placebo. After a period of wash-up all 8 subjects of Cohort S4 received a fat-breakfast and a 750 mg dose of ACI-3024.

The total duration of study participation for each subject (from Screening through Follow-up visit) was approximately 37 days, except for subjects in the food-effect cohort (Cohort S4), for whom the total duration was up to approximately 44 days.

TABLE 1
Subject Population and Treatment Details by Cohort for SAD part

| | | | | | | |
|---|---|---|---|---|---|---|
| | Healthy | | | | | No. of |
| | Subjects | | | | | Subjects |
| Cohort | Per Cohort | Sex | Age Group | Ethnicity | Treatment | (Per Cohort) |

Part I (40 subjects)

| | | | | | | |
|---|---|---|---|---|---|---|
| S1 | 8 | Male | Non-elderly | Non-Japanese | ACI-3024 | 6 |
| | | | | | Placebo | 2 |
| S2 to S5 | 8 | Male | Non-elderly | Japanese | ACI-3024 | 3 |
| | | | | | Placebo | 1 |
| | | | | Non-Japanese | ACI-3024 | 3 |
| | | | | | Placebo | 1 |

Abbreviations: SAD = single ascending dose.
Note:
Non-elderly = 20 to 59 years old;
elderly = 60 to 80 years old.
S1 = 25 mg dose or placebo,
S2 = 100 mg dose or placebo,
S3 = 300 mg dose or placebo,
S4 = 750 mg dose or placebo (in this cohort, all subjects received also a second dose of study treatment in fed state which was ACI-3024 750 mg),
S5 = 1500 mg dose or placebo.

Results/Conclusions:

Single Ascending Dose (SAD)

40 non-Japanese and non-elderly male Japanese healthy subjects were enrolled in the first part of the study (five subsequent single ascending dose cohorts, including a food effect cohort, with eight subjects in each). Overall, 30 subjects received one single dose of ACI-3024 at different doses and 10 subjects received placebo in single ascending dose cohorts S1 to S5. Eight subjects in the food-effect cohort participated in two subsequent treatment periods (with fasted and fed status), and received either a second identical oral dose of ACI-3024 or one dose of placebo and one dose of ACI-3024.

A total of 13 (32.5%) subjects reported a total of 18 AEs (5 AEs in subjects on placebo; respectively 2, 0, 3, 6 and 2 AEs in subjects on 25 mg, 100 mg, 300 mg, 750 mg (fed and fed states) and 1500 mg dose). Dry throat, headache, and nasopharyngitis were the most frequently reported AEs and were each reported by 2 subjects. All AEs were mild in severity. Most AEs were resolved or resolving by the end of the study. There were no SAEs or AEs leading to discontinuation from the study. There were no clinically significant observations in vital signs, electrocardiogram (ECG) values, or in physical or neurological examinations. No subjects withdrew from the study, and there were no serious adverse events (SAEs) reported. Overall, ACI-3024 was considered safe and well tolerated in the SAD study, up to the highest administered dose of 1500 mg.

Multiple Ascending Dose (MAD) Part:

This part was conducted in elderly non-Japanese healthy subjects (MAD HV1 to MAD HV3 cohorts) and in non-elderly Japanese and non-Japanese healthy male subjects (MAD HV4 cohort).

The healthy subject part of Part III was a multiple dose, sequential group study. Overall, 30 healthy elderly male or female non Japanese subjects are being studied in 3 cohorts (Cohorts MAD HV1 to MAD-HV3), with each cohort consisting of 10 subjects. A cohort of 10 healthy non-elderly male Japanese and non-Japanese subjects were enrolled (MAD HV4) to collect safety, tolerability, PK, and PD biomarker data and to enable direct comparisons between non-elderly Japanese and non-Japanese subjects after multiple doses. The dose used in this cohort is based on previously conducted cohorts in non Japanese elderly healthy subjects to assess the effect of multiple doses in non-elderly male Japanese subjects compared to non-elderly non-Japanese male subjects. The MAD HV4 cohort was dosed in parallel with the MAD HV3 cohort.

Cohorts MAD-HV1 to MAD-HV3 comprised 10 elderly non-Japanese male or female subjects, 8 were treated with ACI-3024 and 2 were being given placebo. Cohort MAD-HV4 comprised 5 non-elderly Japanese subjects (4 treated with ACI-3024 and 1 given placebo) and 5 non-elderly Japanese subjects (4 treated with ACI-3024 and 1 given placebo). Only subjects from MAD HV3 cohort underwent CSF samplings to explore the CSF inflammatory markers, CSF PK, and CSF PD of ACI-3024. For all subjects, dosing was on Days 1 to 14, inclusive, and a final single dose administration occurred on Day 14. Dose in cohort MAD HV1 was 500 mg of ACI-3024 or placebo, QD, fasted. Dose in cohorts MAD HV2 and MAD HV4 was 500 mg of ACI-3024 or placebo, BID (“bis in die” ie., twice a day), fed. Dose in cohort MAD HV3 was 1400 mg of ACI-3024 or placebo, QD (“quaque die” (once a day), fed.

TABLE 2
Subject Population and Treatment Details by Cohort for MAD part:

| | Healthy | | | | | No. of |
|---|---|---|---|---|---|---|
| | Subjects | | Age | | | Subjects |
| Cohort | Per Cohort | Sex | Group | Ethnicity | Treatment | (Per Cohort) |
| MAD-HV1 to | 10 | Male or | Elderly | Non- | ACI-3024 | 8 |
| MAD-HV3 | | female | | Japanese | Placebo | 2 |
| MAD-HV4 | 10 | Male | Non- | Japanese | ACI-3024 | 4 |
| | | | elderly | | Placebo | 1 |
| | | | | Non- | ACI-3024 | 4 |
| | | | | Japanese | Placebo | 1 |

Total subjects in Part IIIa: 40
Abbreviations:
MAD = multiple ascending dose.
Note:
Non-elderly = 20 to 59 years old;
elderly = 60 to 80 years old.
MAD-HV1 = 500 mg QD or placebo,
MAD-HV2 = 500 mg BID or placebo,
MAD-HV3 = 1400 mg QD or placebo,
MAD-HV4 = 500 mg BID or placebo.
In MAD-HV1, subjects were fasted on Days 1 and 14 and fed on Days 2-13, subjects were fed during the whole treatment duration on other MAD cohorts.

Results/Conclusions:

In MAD cohorts, a total of 29 (72.5%) subjects reported a total of 89 AEs. Headache and rashes were the most frequently reported AEs. Most AEs were mild (78 AEs) or moderate (9 AEs) in severity. Of 89 AEs, the investigator considered 33 events to be related or possibly related to study drug, including 2 serious adverse events of liver injury possibly related to study drug. Most all-causality AEs were resolved or resolving by the end of the study. In MAD cohorts, the compound did not show SAEs at a dose below 1000 mg/day.

Pharmacokinetics in SAD and MAD Studies

ACI-3024 was rapidly absorbed in the fasting state following single or multiple doses, with median time of maximum observed plasma concentration (Tmax) at 1.25 hours (75 min) to 2.50 (150 min) hours postdose. Food reduced the rate of absorption and increased overall exposure by approximately 3 fold indicating a beneficial effect of administering ACI-3024 during meals. Administration of drugs during meals is preferable as it is an easier and more convenient option for patients.

ACI-3024 was steadily absorbed following multiple dose administrations in the fed state. Statistical assessment indicated an overall dose-proportional increase in AUC0-tlast and AUC0-∞ with a subproportional increase in Cmax. A linear dose proportional increase in exposure is a preferable pharmacokinetic property which enables a better control of exposure in the patient population.

Geometric mean t_{1/2} estimates following single dose administration were between 27.7 hours and 60.5 hours, and when calculable following 14 days of multiple dosing, the geometric mean t_{1/2} was between 47.5 hours and 101 hours. There was evidence of accumulation following multiple dosing with either a QD or BID dosing regimen, consistent with the long half-life.

CSF concentrations, determined in elderly subjects following multiple doses of 1400 mg QD (fed), represented between 0.3% and 1.5% of the corresponding plasma concentration. Following multiple oral doses of 1400 mg the pre-dose (Cmin) CSF concentrations were 36.4±13.6 ng/mL (90 nM) at Day 7. Only a small amount of ACI-3024 was excreted in urine. This data suggests that the compound efficiently crosses the blood brain barrier. This suggests that the dose of 300-400 mg/day (such as for example, 350) would provide sufficient CSF exposure (30 nM).

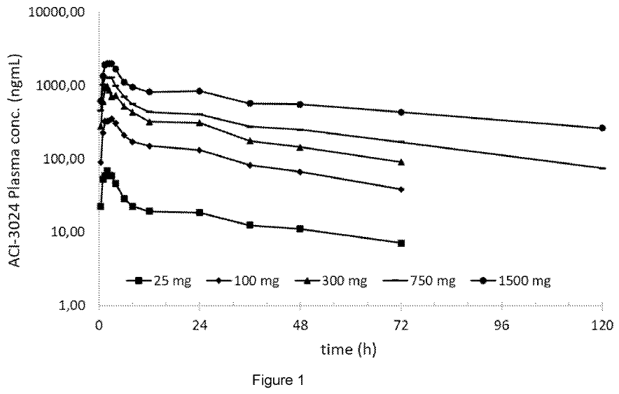

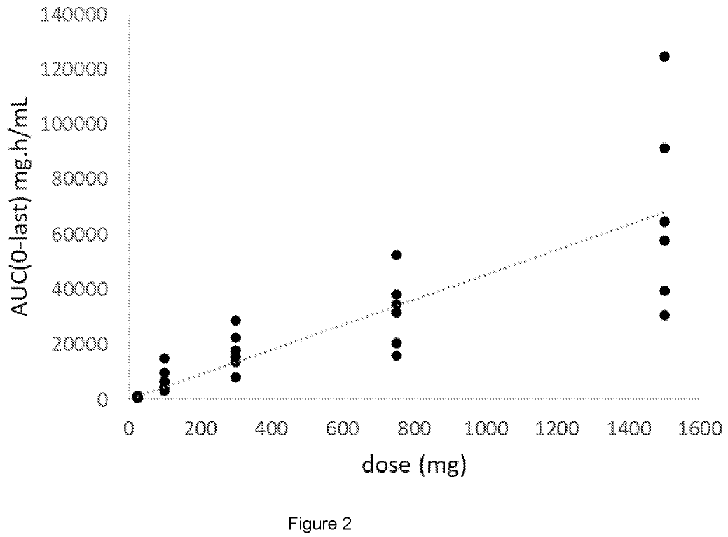

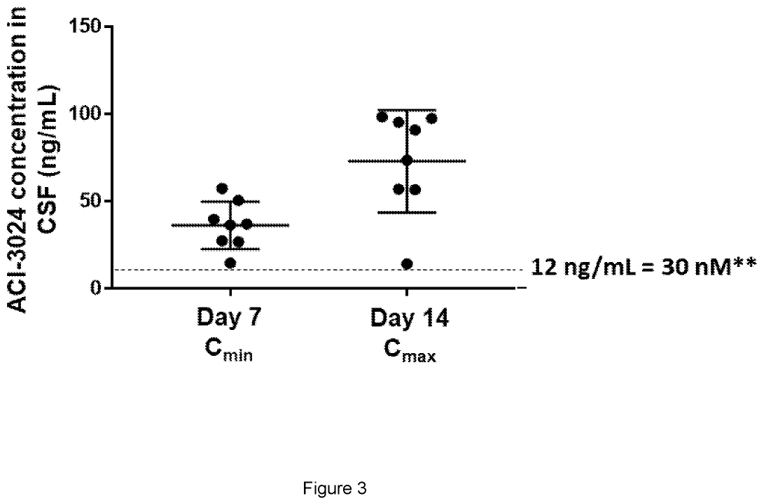

ACI-3024 showed linear, favorable pharmacokinetic and long half-life after oral administration in healthy volunteers (see FIGS. 1 and 2) which is preferable for a chronic treatment in patients. CSF pre-dose concentrations exceeded the target established in animal models of 30 nM (see FIG. 3) which was established from studies of ACI-3024 in the Tg4510 transgenic mouse model which reflects the clinical manifestation of tauopathies.

In summary, the brain target exposure in subjects enrolled in Phase 1 in the MAD study was achieved after administration of ACI-3024 compound.

### Example 2

4-Week Toxicity Study in Mice Followed by 2 Weeks of Recovery

Clinical Study Protocol

This study was conducted to evaluate the potential toxicity of ACI-3024 when given daily by oral gavage to CD1 mice for 4 weeks, followed by a 2-week recovery period. Doses of 100, 300 and 1000 mg/kg were administered by p.o. to 10 animals/sex/group daily. The control animals were administered vehicle. The dose volume was 10 mL/kg for all groups.

Clinical signs, body weights, food consumption, ophthalmology, clinical pathology were evaluated. For each sex/group, a complete necropsy was conducted on 10 animals at the end of the dosing period and on 6 (only control and high dose groups) surviving animals at end of the recovery period.

Results and Conclusions

All animals were exposed to the test item (ACI-3024). Time to maximum plasma concentration was reached between 1 and 6 hours after administration in all groups with the exception of females in the high-dose group for which delayed Tmax (24 h post-administration) was observed, Plasma exposure increased less than dose proportionally over the range of tested doses. In general, higher exposure was observed in females than in males except at 300 mg/kg/day on Day 28. The difference in exposure was more marked at the beginning of the study (Day 1), while at Day 28 exposure in male and female animals was comparable. A slight ACI-3024 accumulation was observed after repeated administrations (Day 1 vs. Day 28).

TABLE 3
Mean Toxico Kinetic (TK) parameters of ACI-3024 following oral
administration of ACI-3024 in male and female Swiss (CD-1) mice

| | Dose level | | Tₘₐₓ | Cₘₐₓ | AUC₀₋ₜ |
|---|---|---|---|---|---|
| Period | mg/kg | Sex | h | ng/mL | h*ng/mL |
| Day 28 | 300 | F | 4 | 23372 | 319652 |
| Day 28 | 300 | M | 4 | 28275 | 385964 |
| Day 28 | 300 | Average M/F | 4 | 25824 | 352808 |

At 100 mg/kg/day, there were no clinical signs, or changes in mean body weight, body weight gain, food consumption, ophthalmology, clinical pathology parameters or pathology.

At 1000 mg/kg/day, only one female animal was prematurely euthanized on the basis of the poor clinical condition on Day 9 with no macroscopic findings. In some surviving animals, clinical signs such as thin appearance, piloerection, hunched posture, dyspnea and pallor of the extremities in females appeared mainly from Day 4 onwards.

At 300 mg/kg/day, piloerection and hyperactivity were occasionally observed in a few animals. No relevant changes in mean body weight, body weight gain or food consumption were noted. No significant changes were recorded in the ophthalmological or in clinical pathology parameters. At pathology, there were no organ weight changes, macroscopic findings or adverse findings attributable to ACI-3024 administration within the pancreas (atrophy, exocrine; present in females), spleen (EMH, increased; present in females), and vagina (mucification of the vagina, suggestive of premature senescence; present in females).

Under the experimental conditions of the study, the No Observed Adverse Effect Level (NOAEL) in mice given ACI-3024 for 4 weeks was established at 300 mg/kg/day. At this dose level, the mean systemic exposure of test item in terms of Cmax and AUC were respectively, 28′275 ng/mL and 385′964 h*ng/mL for males and 23′372 ng/mL and 319′652 h*ng/mL for females. The average Cmax and AUC between males and females were 25824 ng/mL and 352808 h*ng/mL, respectively, see table 3.

### Example 3

4-Week Toxicity Study in Mini-Pigs Followed by 2 Weeks of Recovery

Clinical Study Protocol

This study was conducted to evaluate the potential toxicity of ACI-3024 in a non-rodent species when given daily to Gottingen mini-pig for 4 weeks, followed by a 2-week recovery period.

Doses tested, 50, 150 and 450 mg/kg were given by p.o. to 3 animals/sex/group daily. The control animals were administered vehicle. The dose volume was 5 mL/kg for all groups.

Clinical signs, body weights, food consumption, electrocardiography, ophthalmology and clinical pathology were evaluated. For each sex/group, a complete necropsy was conducted on 3 animals at the end of the dosing period and on 2 (only control and high dose groups) surviving animals at end of the recovery period.

Toxico Kinetic (TK) was evaluated on all animals on study Day 1 and Day 28 at different time points.

Results and Conclusions

A minimal test item-related reduction in body weight gain was observed in both sexes during the treatment phase, when compared with concurrent controls animal. Overall the body weight changes in minipigs were not considered adverse. Neither electrocardiographic nor ophthalmic alterations occurred at the study protocol. In respect of histopathology examination, there were no treatment-related microscopic findings in study animals. Time to maximum plasma concentration was reached between 1 and 6 hours after administration. Plasma exposure increased approximately dose-proportionally in the range of the administered doses excepted in males in which a less dose proportional increase, along with high variability, was observed on Day 1.

TABLE 4
Mean Toxico Kinetic (TK) parameters of ACI-3024 following
oral administration of ACI-3024 in male and female minipigs

| | Dose level | | Tₘₐₓ | Cₘₐₓ | AUC₀₋ₜ |
|---|---|---|---|---|---|
| Period | mg/kg | Sex | h | ng/mL | h*ng/mL |
| Day 28 | 450 | F | 4 | 7348 | 101191 |
| Day 28 | 450 | M | 6 | 12323 | 232774 |
| Day 28 | 450 | Average M/F | 5 | 9836 | 166983 |

In conclusion daily oral (gavage) treatment to Gottingen mini-pigs with ACI-3024 at dosages up to 450 mg/kg for 4 weeks was well tolerated.

Under the experimental conditions of the study, an adverse effect level was not established and the No Observed Adverse Effect Level (NOAEL) was considered as 450 mg/kg, the highest dose tested. At this dose level, the mean systemic exposure of test item in terms of Cmax and AUC were respectively, 12′323 ng/mL and 232′774 h*ng/mL for males and 7′348 ng/mL and 101′191 h*ng/mL for females. The average Cmax and AUC between males and females were 9836 ng/mL and 166983 h*ng/mL, respectively.

### Example 4

Central Nervous System Evaluation in Mice

Clinical Study Protocol

This study was conducted to evaluate the potential modifications of central nervous system (CNS) activity by ACI-3024 when given as single dose to CD1 mice.

Doses of 100, 300 and 750 mg/kg were administered by oral gavage to 8 females/group. The control animals were administered vehicle. The dose volume was 10 mL/kg for all groups. Approximately 2, 4, 6 and 24 hours after the treatment, each mouse was subjected to a functional observation battery in order to investigate possible treatment-induced effects on the CNS or in any of its domains: neurologic, autonomic and behavioral.

Results and Conclusions

In conclusion, under the experimental conditions of the study, a single oral administration of ACI-3024 at 100, 300 or 750 mg/kg had no effect on Central Nervous System function in this observational study.

### Example 5

Respiratory Function Evaluation in Mice

Clinical Study Protocol

This study was conducted to evaluate the potential modifications of respiratory function by ACI-3024 when given as single dose to CD1 mice.

Doses of 100, 300 and 750 mg/kg were administered by oral gavage to 8 females/group. The control animals were administered vehicle. The dose volume was 10 mL/kg for all groups. Animals underwent a whole body plethysmography carried out starting 30 minutes before dosing up to 5 hours after; moreover, a subsequent 1-hour registration was conducted 24 hours post dose. Respiratory parameters, including respiratory rate, peak inspiratory and expiratory flows, enhanced pause and minute and tidal volumes, were recorded before dosing (mean of −15, −10, and −5 minutes) and 30, 60, 90, 120, 150, 180, 240, 300 and 1440 minutes after the end of administration.

Results and Conclusions

In conclusion, in this study, respiratory parameters were continuously evaluated by whole body plethysmography for up to 5 hours with an additional single point at 24 hours in conscious unrestrained mice female. Under the experimental conditions of the study, a single oral administration of the test item ACI-3024 at 100, 300 and 750 mg/kg did not exert effect on respiratory parameters.

### Example 6

Cardiovascular Evaluation in Mini-Pig

Clinical Study Protocol

This study was conducted to evaluate the potential effects on the cardiovascular system of ACI-3024 when given as single dose to telemetrized Gottingen mini-pig.

Doses of 0, 50, 150 and 450 mg/kg were administered by oral gavage to 4 males/group. The dose volume was set at 5 mL/kg. The study was a randomized cross over design which consisted of four phases separated by seven days of wash out. In each phase each animal received as single administration of one selected doses. Through the phases each animal received all the dose levels once. ECG (heart rate, duration of the PR, QRS, QT, QTcF (QT interval corrected by Fridericia) and RR intervals (millisecond; ms), blood pressure (systolic, diastolic, and mean arterial pressure) and body temperature parameters through a transmitter implanted into the animals were continuously recorded starting from 30 minutes before dosing up to 20 hours after dose. Different timepoints during the recording period were sampled (−30 min (before dose) and 1 h, 2 h, 4 h, 6 h, 8 h, 12 h and 20 h (after dose)), per each timepoint 5-minute segment was used for study purposes.

Results and Conclusions

In conclusion, under experimental conditions of the present study, there was no effect of ACI-3024 administered orally (dose levels 0, 50, 150 and 450 mg/kg) to Gottingen mini-pigs on cardiovascular parameters and body temperature.

### Example 7

ACI-3024 Bioavailability in Bama Mini-Pig Data Orally Administered as a Capsule, Dissolved in Water or in 3% TPGS Suspension

Protocol: Prototype 1 Intra-granular composition of capsules were prepared by mixing 200 mg of ACI-3024 corresponding to 52.6% w/v, in Microcrystalline cellulose Avicel PH102 (14.5% w/v), Lactose monohydrate (Granulac 200, 422.9% w/v), Hydroxy Propyl Cellulose HPC (Klucel LF, 2% w/v), Crospovidone (2.5% w/v), and Purified water (QS % w/w of drymix). Prototype 1 Extra-granular composition of capsules were prepared by mixing Microcrystalline cellulose Avicel PH102 (2.0% w/v), Crospovidone (2.5% w/v), Aerosil pharma200 (0.5% w/v), and Magnesium stearate (0.5% w/v).

QS means Quantity Sufficient

The PK profile of ACI-3024 was evaluated in male Bama mini-pigs after oral administration (gavage) of Protototype 1 capsules. The following formulations were tested: (i) Prototype 1 capsule administered directly, using the pill gun, placing one capsule per animal beyond the root of tongue; swallowing was facilitated by massaging the animal's throat and by administering 20 mL of water, (ii) Prototype 1 capsule dissolved in 20 mL of water 1 h before administration, (iii) the content of Prototype 1 capsule suspended in 20 mL of 3% (30 mg/ml) TPGS in water, yielding a homogenous opaque suspension.

Animals were dosed fasted and food was returned 2 h post-dosing.

Bama mini-pigs weight was measured at administration. ACI-3024 was effectively administered at 17.5, 12.6 and 10.1 mg/kg, respectively for Prototype 1 capsule, capsule dissolved in water and capsule suspended in 3% TPGS in water.

To evaluate the bioavailability and PK profile, blood was collected from peripheral veins at 15′, 30′, 1, 2, 4, 6, 8, 12, 24, 48, 72 h. Plasma was derived and the concentrations of ACI-3024 were measured by LC MS/MS using the acetonitrile-extracted plasma samples.

PK profiles were derived. Additionally, absolute bioavailability (% F) was calculated using the data from intravenously administered animals. Table 5 shows the main PK parameters and the % F values.

Results and Conclusions

TABLE 5

| | Treatment groups | | |
|---|---|---|---|
| | | | Capsule |
| | Prototype 1 | Capsule | content in 3% |
| PK Parameters | capsule | in water | TPGS in water |
| Administered | 17.52 | 12.62 | 10.11 |
| dose (mg/kg) | | | |
| Cmax (ng/mL) | 194 | 220.3 | 244 |
| % CV on Cmax | 61.2 | 10.7 | 26.6 |
| AUC_{0-inf} (ng · h/mL) | 5822.7 | 3760.8 | 6126.4 |
| T_{1/2} (h) | 6.29 | 9.81 | 8.97 |
| Bioavailability (%) | 32.5 | 35.0 | 88.0 |

Among the tested formulations, the content of the capsule Prototype 1 suspended in 3% TPGS shows the highest bioavailability (88%) with low variability and long half-life (T_{1/2}).

1. A method of treating, preventing, or alleviating a disorder or an abnormality associated with Tau protein aggregates or a tauopathy comprising administering said a compound ACI-3024 in an amount of about 25 mg/day to about 1500 mg/day to a mammal subject in need thereof.

2. The method according to claim 1 wherein the compound is given as single dose without inducing a serious adverse event in the subject.

3. The method according to claim 1 wherein the compound is given multiple times in a total amount below 1000 mg/day without inducing a serious adverse event in the subject.

4. The method according to claim 1 wherein ACI-3024 is administered at the amount up to about 1000 mg/day or about 500 mg/day.

5. The method according to claim 1, wherein the subject is a human, and wherein the compound ACI-3024 is characterized by a plasma half-life of at least 24 hours.

6. The method according to claim 1, comprising administering the compound ACI-3024 to the mammal subject at an amount that results in a Cmin concentration above or equal to 30 nM of the compound in CSF of the subject.

7. The method according to claim 1, wherein the compound amount is up to or equal to about 300, 350, or 400 mg/day.

8. The method according to claim 1, wherein the compound amount is from about 300 or 400 mg/day.

9. The method according to claim 1, wherein the compound is in the range of 300 mg/day to 350 mg/day.

10. The method according to claim 1, wherein the daily dose is divided into unit dose(s) to be administered one, two or three times per day.

11. The method according to claim 1, wherein the mammal subject is a human subject.

12. The method according to claim 1, wherein ACI-3024 is administered orally.

13. The method according to claim 12 wherein said ACI-3024 is administered in the form of tablet, capsule, solution or suspension.

14. The method according to claim 1, wherein the disorder or the abnormality associated with Tau protein aggregates or a tauopathy is selected from Alzheimer's disease (AD), familial AD, PART (Primary Age-Related Tauopathy), Creutzfeldt-Jacob disease, dementia pugilistica, Down's Syndrome, Gerstmann-Straussler-Scheinker disease (GSS), inclusion-body myositis, prion protein cerebral amyloid angiopathy (PrP-CAA), traumatic brain injury (TBI), amyotrophic lateral sclerosis (ALS), Parkinsonism-dementia complex of Guam, non-Guamanian motor neuron disease with neurofibrillary tangles, argyrophilic grain disease (AGD), corticobasal degeneration (CBD), diffuse neurofibrillary tangles with calcification, frontotemporal dementia with Parkinsonism linked to chromosome 17 (FTDP-17), FTLD-MAPT (frontotemporal lobar degeneration caused by a MAPT gene mutation), frontotemporal lobar degeneration with predominant Tau pathology (FTLD-Tau), Hallervorden-Spatz disease, multiple system atrophy (MSA), Niemann-Pick disease type C, pallido-ponto-nigral degeneration, Pick's disease (PiD), progressive subcortical gliosis, progressive supranuclear palsy (PSP), subacute sclerosing panencephalitis, tangle predominant dementia, postencephalitic Parkinsonism, myotonic dystrophy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy including Lafora disease, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD and Huntington's disease.

15. The method according to claim 14 wherein the disorder or the abnormality associated with Tau protein aggregates or a tauopathy is selected from Alzheimer's disease (AD).

16. The method according to claim 14 wherein the disorder or the abnormality associated with Tau protein aggregates or a tauopathy is selected from progressive supranuclear palsy (PSP).

17. The method according to claim 14 wherein the disorder or the abnormality associated with Tau protein aggregates or a tauopathy is selected from frontotemporal lobar degeneration with predominant Tau pathology (FTLD-Tau).

18. The method according to claim 14 wherein the disorder or the abnormality associated with Tau protein aggregates or a tauopathy is selected from frontotemporal lobar degeneration caused by a MAPT gene mutation (FTLD-MAPT).

19. The method according to claim 14 wherein the disorder or the abnormality associated with Tau protein aggregates or a tauopathy is selected from corticobasal degeneration (CBD).

20. The method according to claim 14 wherein the disorder or the abnormality associated with Tau protein aggregates or a tauopathy is selected from Pick's disease (PiD).

21. The method according to claim 14 wherein the disorder or the abnormality associated with Tau protein aggregates or a tauopathy is Lafora Disease.

22-25. (canceled)

26. The method according to claim 1, comprising preventing the formation of Tau aggregates and/or of inhibiting Tau aggregation.

27. The method according to claim 1, comprising interfering intracellularly with Tau aggregates.

28. The method according to claim 1, comprising reducing Tau misfolding and hyperphosphorylation in vivo, the method comprising administering an amount of the compound ACI-3024 as defined in any one of claims 1 to 10 to a mammal subject in need thereof.

29. The method according to claim 1, comprising reducing neuroinflammatory markers comprising administering an amount of the compound ACI-3024 as defined in any one of claims 1 to 10 to a mammal subject in need thereof.

30. A pharmaceutical composition comprising compound ACI-3024 in an amount of about 25 mg/day to about 1500 mg/day.

31. The pharmaceutical composition according to claim 30, comprising a Vitamin E Polyethylene Glycol Succinate (TPGS), such that the pharmaceutical composition can be administered orally to a mammal subject in need thereof.

32. A method for obtaining the pharmaceutical composition as defined in claim 31, wherein a solution comprising 3% TPGS/water is mixed with a solid form of ACI-3024 for obtaining a suspension.

33. The pharmaceutical composition according to claim 31, wherein the mammal subject in need thereof is a human.
